(19) United States
(12) Patent Application Publication
(10) Pub. No.: US 2017/0106033 A1
(43) Pub. Date: Apr. 20, 2017

(54) SEMEN CASSIAE-CONTAINING TEA BAG AND PREPARATION METHOD THEREOF

(71) Applicant: Jiangsu Daocheng Biotechnology Co., Ltd, Yancheng (CN)

(72) Inventors: Zhengguo Gu, Yancheng (CN); Yangchun Chen, Yancheng (CN); Wei Zhang, Yancheng (CN); Yilin Xie, Yancheng (CN)

(21) Appl. No.: 15/182,608

(22) Filed: Jun. 15, 2016

(30) Foreign Application Priority Data
Oct. 16, 2015 (CN) 201510669838.4

(51) Int. Cl. A61K 36/48; A61K 36/605; A61K 36/738; A61K 36/484; B65D 85/808; A61K 8/97; A61Q 19/08; A61K 9/00; A23F 3/14; A61K 36/287; A61K 36/78

(52) U.S. Cl. CPC A61K 36/48; A61K 36/287; A61K 36/605; A61K 36/738; A61K 36/484; A61K 36/78; A61K 8/97; A61Q 19/08; A61K 9/0053; A23F 3/14; B65D 85/808; A23L 1/3002; A61K 2800/5922; A61K 2800/87; A61K 2800/92; A23V 2002/00

(57) ABSTRACT

The invention discloses a Semen Cassiae-containing tea bag and preparation method thereof. The Semen Cassiae-containing tea bag comprises Semen Cassiae, chrysanthemum, lotus leaf and folium mori. The preparation method comprises the following steps: (1) sorting; (2) grinding; (3) sieving; (4) sterilizing: placing a sieved powdery mixture into a material tray, and performing ozone sterilizing for 30 minutes, wherein the material thickness in the material tray does not exceed 5 cm; (5) weighing; (6) mixing and (7) packing. The tea bag has the beneficial effects that the tea bag is rich in color, pure and mild in taste, particularly suitable for female consumers, is simple and convenient, and has the effects of diuresis and bowel relaxing, heat clearing, detoxifying, resisting aging and beautifying, has no any side effects for a human body after drinking, complies with a tea drinking tradition of people, solves the problem of single flavor of tea leaves and bitter taste caused by direct drinking of the Semen Cassiae and is more obvious in the effects after drinking for a long term, and is easily accepted by the female consumers, and the cost is saved.

### CROSS-REFERENCE TO RELATED APPLICATIONS

This application is a continuation of, and claims priority to, Chinese Patent Application No. 201510669838.4 with a filing date of Oct. 16, 2015. The content of the aforementioned application, including any intervening amendments thereto, is incorporated herein by reference.

### TECHNICAL FIELD

The invention relates to the field of tea bag in particular to a Semen Cassiae-containing tea bag and preparation method thereof.

### BACKGROUND OF THE INVENTION

Semen Cassiae is a Chinese herbal medicine with multivitamin, rich amino acid and carbohydrate. It has effects of clearing liver fire, tonifying kidney, improving eyesight and relaxing bowel. Chrysanthemum, lotus leaf and folium mori have been made to various substantial teas in modern scientific study, which has effects of ageing resisting and beautifying. Most people have habit of drinking tea, however, they are accustomed to use the substitute of tea, which is single in taste, bad in flavor because of processing technique and high cost, so most consumers still cannot accept it.

Chinese patent CN101653472A discloses a preparation method of Semen Cassiae tea bag, in the method, Semen Cassiae is adopted as raw material, and the method comprises the following steps: selection, roasting and aroma extraction, smashing, sieving and granulator straightening and packaging; wherein granulator straightening is carried out by a sieve with 10˜65 meshes. The prepared Semen Cassiae tea bag by this method is low in effective composition content, bad in flavor and poor in taste.

So it is necessary to study a new Semen Cassiae-containing tea bag for improving the application amount of Semen Cassiae tea bag in consumers.

### SUMMARY OF THE INVENTION

The purpose of the invention is to provide a Semen Cassiae-containing tea bag tea bag with good taste and the effects of diuresis and bowel relaxing, heat clearing, detoxifying, aging resisting and beautifying.

To solve above technical problems, the technical proposal of the invention is the tea bag comprises Semen Cassiae, chrysanthemum, lotus leaf and folium mori.

In above technical proposal, Semen Cassiae is a medicine with faint breath and slight bitter taste, the Semen Cassiae full of particles and green and brown color is the best. Its chemical components include emodin, chrysophanol, physcion, obtusin, obtusifolin, and glycosides, with effects of clearing heat, improving eyesight, relaxing bowel and losing weight, Semen Cassiae can be used for the treatment of headache, dizziness, blurring vision, liver, kidney, large intestine, wind-heat red eye, glaucoma, night blindness, hypertension, hepatitis, ascites due to cirrhosis and habitual constipation; chrysanthemum has effects of removing wind and clearing heat, calming liver and improving eyesight, and clearing heat and removing toxicity, using chrysanthemum cooperatively with Semen Cassiae, the effects of Semen Cassiae can be better, and chrysanthemum is bitter and cold, which can control the efficacy of Semen Cassiae effectively and improve the taste of tea; the large, trim and green lotus leaf is the best, the mixture in tea bag is fresh in color and delicate in flavor with lotus leaf, lotus leaf has effects of relieving summer heat and dampness, tonifying spleen, and dissipating stasis and stopping bleeding, using the lotus leaf cooperatively with Semen Cassiae can be more diuretic, however, the amount need to be controlled for preventing diarrhea; folium mori is dark green, the tender leaves are thin, it has effects of improving the immune capability, resisting ageing and beautifying; compared with prior art, the invention has the beneficial effects that the tea bag is rich in color, pure and mild in taste, particularly suitable for female consumers, is simple and convenient, and has the effects of diuresis and bowel relaxing, heat clearing, detoxifying, aging resisting and beautifying, has no any side effects for a human body after drinking, complies with a tea drinking tradition of people, solves the problem of single flavor of tea leaves and bitter taste caused by direct drinking of the Semen Cassiae, and is more obvious in the effects after drinking for a long term, and is easily accepted by the female consumers, and the cost is saved.

A further improvement is characterized in that the tea bag comprises following raw materials in parts by weight: Semen Cassiae 100˜300 parts, chrysanthemum 50˜150 parts, lotus leaf 200˜400 parts and folium mori 50˜180 parts; wherein the lotus leaf can cover the bitter taste of Semen Cassiae and chrysanthemum because of its green color and fresh flavor, the highest weight ratio is 2/3, but the amount need to be controlled for preventing diarrhea.

A further improvement is characterized in that the tea bag also comprises following raw materials in parts by weight: rose 50˜150 parts, licorice 50˜150 parts, and herba houttuyniae 50˜180 parts.

The tea bag is better in color and stronger in scent because of rose, besides, it has effects of regulating flow of qi, activating blood and convergence, using rose cooperatively with lotus leaf can motivate the function of beautifying, but the amount need to be controlled for preventing diarrhea; the sweet taste of licorice helps for covering the bitter taste of other materials; and the acrid flavor of herba houttuyniae with other materials can improve the taste of tea bag, besides, herba houttuyniae has effects of antibiosis, antivirus and organism immunity improving.

A further improvement is characterized in that the rose is Rosa rugosa cv.Plena; compared with other varieties, Rosa rugosa cv.Plena is more suitable for tea bag.

A further improvement is characterized in that the tea bag comprises following raw materials in parts by weight: Semen Cassiae 150 parts, chrysanthemum 50 parts, lotus leaf 220 parts, folium mori 100 parts, Rosa rugosa cv.Plena 75 parts, licorice 80 parts and herby houttuyniae 180 parts

Based on extensive research, the effects of bowel relaxing, heat clearing and detoxifying, aging resisting and beautifying are the best using above weight ratio, and through contrast experiment, the tea bag with this ratio of parts by weight is more easily accepted by female consumers and the taste is better.

Another purpose of the invention is to provide a preparation method for Semen Cassiae-containing tea bag with good taste and the effects of diuresis and bowel relaxing, heat clearing, detoxifying, aging resisting and beautifying.

The technical proposal of the invention is the preparation method for Semen Cassiae-containing tea bag comprises the following steps:

(1) sorting: taking fresh raw materials and cleaning, drying with 30˜50 DEG C hot wind, and removing visible impurities, the impurities content does not exceed 5% weight ratio:

taking fresh raw materials and cleaning based on the need of prescription, exposing under the blazing sun is not allowed because it easily leads to incompleteness of materials and great loss, and drying in the shade is also not allowed because it easily leads to mouldiness of material and affects the quality of tea; drying with 30˜50 DEG C hot wind can save time and keep the completeness of material, however, it does not be dried for a long time, 3˜5 minutes are enough to dry the surface moisture;

(2) grinding: grinding the raw materials after step (1) by 40 meshes and obtaining a powdery mixture;

(3) sieving: sieving the powdery mixture, so that 95% weight ratio of the mixture can reach 40 meshes for brewing up the effective ingredients of materials, and no more than 5%˜10% weight ratio of the mixture reach 60 meshes for preventing the turbid tea water;

(4) sterilizing: placing a sieved powdery mixture into a material tray, and performing ozone sterilizing for 30 minutes, wherein the material thickness in the material tray does not exceed 5cm;

(5) weighing: weighing according to weight ratio of each material bag;

(6) mixing: mixing for 30 minutes in three dimensional mixer, so that obtained materials in above steps can be adequately mixed; and

(7) packing: using the integrative machine of tea bag to pack by direct forming method, each packing unit is 2.5 g.

A further improvement is characterized in that the packaged tea bag comprises following raw materials in parts by weight: Semen Cassiae 100˜300 parts, chrysanthemum 50˜150 parts, lotus leaf 200∥400 parts, folium mod 50˜180 pans, Rosa rogosa cv.Plena 50˜150 parts, licorice 50˜150 parts and herbs houttuyniae 50˜180 parts.

A further improvement is characterized in that a vacuum roasting step is added between the step (3) sieving and step (4) sterilizing, in the vacuum roasting, vacuum degree is 0.1˜1 MPa, and temperature is 50˜60 DEG C.

The purpose of vacuum roasting is to prevent the oxidation of effective ingredients and preserve the effective ingredients as much as possible: besides, the materials through vacuum roasting are more pure and mild in taste and more easily accepted by consumers.

A further improvement is characterized in that a freezing step is added between the step (1) sorting and step (2) grinding, the freezing step is to freeze the sorted fresh raw materials in step (1) for 3 h under minus 10˜minus 6 DEG C; the materials will be hardened and easily grinded and the meshes can be smaller, through study, the frozen materials can be more easily dissolved in the process of brewing and reach instant dissolving effect, the tea water is more rich in color, more pure and mild in taste.

A further improvement is characterized in that after vacuum roasting, moisture content of materials is lower than 6%.

### BRIEF DESCRIPTION OF THE DRAWINGS

FIG. 1 is a schematic diagram of an inner bag of Semen Cassiae-containing tea bag according to the invention.

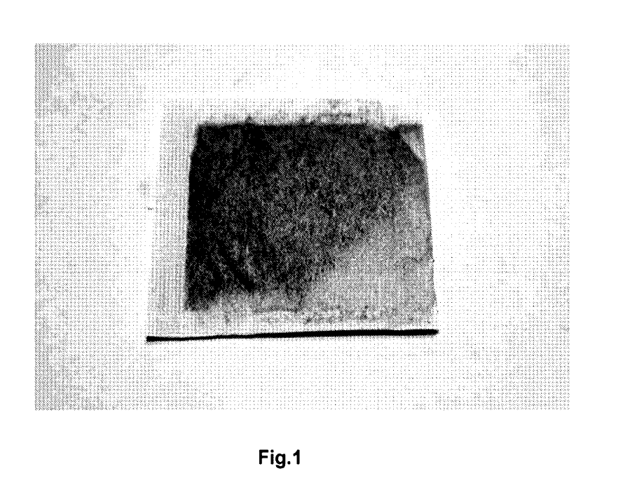

FIG. 2 is a schematic diagram of brewing tea bag in FIG. 1 with boiled water.

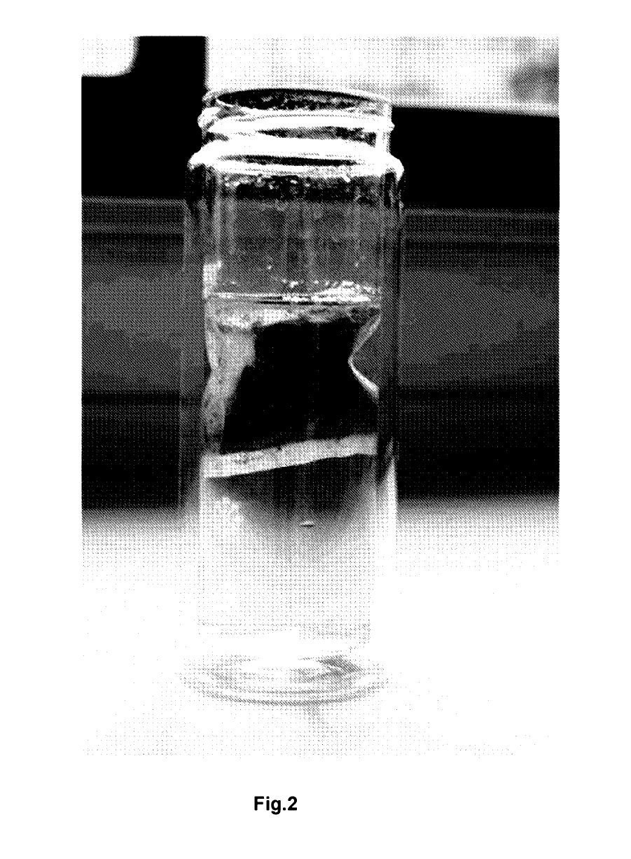

FIG. 3 is a schematic diagram after 5 seconds of brewing tea bag in FIG. 2.

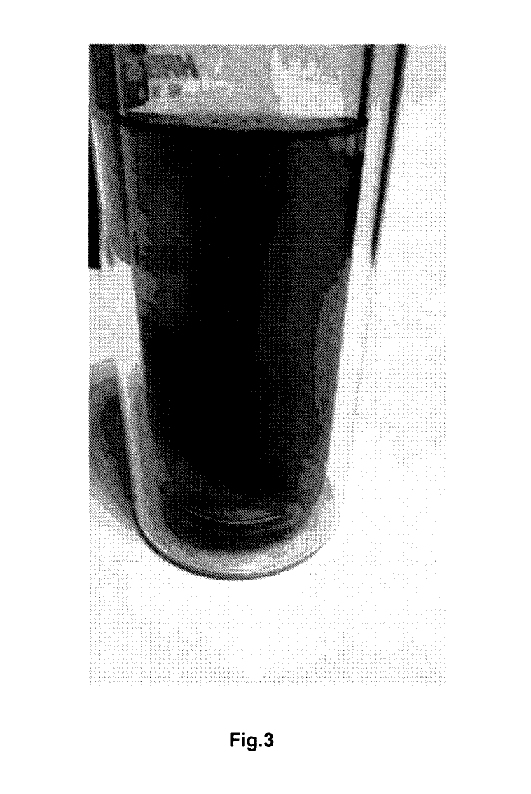

### DETAILED DESCRIPTION OF PREFERRED EMBODIMENTS

### Embodiment 1

a Semen Cassiae-containing tea bag, comprising following raw materials in parts by weight: Semen Cassiae 100 parts, chrysanthemum 50 parts, lotus leaf 200 parts and folium mori 50 parts.

The preparation method of above Semen Cassiae-containing tea bag comprises the following steps:

sorting: taking fresh raw materials of Semen Cassiae, chrysanthemum, lotus leaf and folium neon, and cleaning, drying with 30˜50 DEC C hot wind, and removing visible impurities, the impurities content does not exceed 5% weight ratio;

freezing: freezing the sorted fresh raw material step (1) 3 h under minus 10˜minus 6 DEC C;

grinding: grinding the raw materials after step (1) by 40 meshes and obtaining a powdery mixture;

sieving: sieving the powdery mixture, so that 95% weight ratio of the mixture can reach 40 meshes, and no more than 5%-10% weight ratio of the mixture reach 60 meshes;

sterilizing: placing a sieved powdery mixture into a material tray, and performing ozone sterilizing for 30 minutes, wherein the material thickness in the material tray does not exceed 5 cm;

weighing: weighing according to weight ratio of each material in tea bag:

mixing: mixing for 30 minutes in three dimensional mixer; and

packing: using the integrative machine of tea bag to pack by direct forming method, each packing unit is 2.5 g.

The tea bag can be brewed directly with boiled water, no more than 5 seconds, it is attractively fragrant, pure and mild in taste, and has effects of diuresis and bowel relaxing, heat clearing, detoxifying, aging resisting and beautifying.

### Embodiment 2

the difference with embodiment 1 is the weight ratio of each material: Semen Cassiae 300 parts, chrysanthemum 150 parts, lotus leaf 400 parts and folium mori 180 parts.

The tea bag can be brewed directly with boiled water, no more than 5 seconds, it is attractively fragrant, pure and mild in taste, and has effects of diuresis and bowel relaxing, heat clearing, detoxifying, aging resisting and beautifying, and it is welcomed by testers.

### Embodiment 3

Semen Cassiae 150 parts, chrysanthemum 50 parts, lotus leaf 220 parts and folium mori 100 parts.

The tea bag can be brewed directly with boiled water, no more than 5 seconds, it is attractively fragrant, pure and mild in taste, the color of tea water will be deepen and has effects of diuresis and bowel relaxing, heat clearing, detoxifying, aging resisting and beautifying.

### Embodiment 4

the difference with embodiment 3 is the tea bag also comprises following raw materials in parts by weight: Rosa rugosa cv.Plena 75 parts, licorice 80 parts and herbs houttuyniae 180 parts, and the preparation method is the same with the embodiment 1.

### Embodiment 5

the packaged Semen Cassiae-containing tea bag comprises following raw materials in parts by weight: Semen Cassiae 100 parts, chrysanthemum 50 parts, lotus leaf 200 parts, folium Mori 50 parts, Rosa rugosa cv.Plena 50 parts, licorice 50 parts and herbs houttuyniae 50 parts.

The preparation method for Semen Cassiae-containing tea bag of this embodiment comprises the following steps:

sorting: taking fresh raw materials of Semen Cassiae, chrysanthemum, lotus leaf and folium mori, and cleaning, drying with 30˜50 DEG C hot wind, and removing visible impurities, the impurities content does not exceed 5% weight ratio;

freezing: freezing the sorted fresh raw materials in step (1) for 3 h under minus 10˜minus 6 DEG C

grinding: grinding the raw materials after step (1) by 40 meshes and obtaining a powdery mixture;

sieving: the powdery mixture, so that 95% weight ratio of the mixture can reach 40 meshes, and no more than 5%-10% weight ratio of the mixture reach 60 meshes;

vacuum roasting: in the vacuum roasting, vacuum degree is 0.1˜1MPa, and temperature is 50˜60 DEG C., and moisture content of materials is lower than 6%.

sterilizing: placing a sieved powdery mixture into a material tray, and performing ozone sterilizing for 30 minutes, wherein the material thickness in the material tray does not exceed 5 cm;

weighing: weighing according to weight ratio of each material in tea bag;

mixing: mixing for 30 minutes in three dimensional mixer; and

packing: using the integrative machine of tea bag to pack by direct forming method, each packing unit is 2.5 g.

The tea bag can be brewed directly with boiled water, no more than 5 seconds, it is attractively fragrant, pure and mild in taste, and has effects of diuresis and bowel relaxing, heat clearing detoxifying, aging resisting and beautifying.

### Embodiment 6

the packaged Semen Cassiae-containing tea bag comprises following raw materials in parts by weight: Semen Cassiae 300 parts, chrysanthemum 150 parts, lotus leaf 400 parts, folium mod 180 parts, Rosa rugosa cv.Plena 150 parts, licorice 150 parts and herba houttuyniae 180 parts, and the preparation method is the same with embodiment 5.

The tea bag can be brewed directly with boiled water, no more than 5 seconds, it is attractively fragrant, pure and mild in taste, and has effects of diuresis and bowel relaxing, heat clearing, detoxifying, aging resisting and beautifying.

### Embodiment 7

in this embodiment, the packaged tea bag comprises following raw materials in parts by weight: Semen Cassiae 150 parts, chrysanthemum 50 parts, lotus leaf 220 parts, folium mori 100 parts. Rosa rugosa cv.Plena 75 parts, licorice 80 parts and herba houttuyniae 180 parts, and the preparation method is the same with embodiment 5.

The tea bag can be brewed directly with boiled water, no more than 5 seconds, it is attractively fragrant, pure, and mild in taste, and has effects of diuresis and bowel relaxing, heat clearing, detoxifying, aging resisting and beautifying, compared with embodiment 4, the effect of the tea bag prepared by this embodiment is better.

We claim:

1. A Semen Cassiae-containing tea bag, comprising semen cassia, chrysanthemum, lotus leaf, and folium mori.

2. The Semen Cassiae-containing tea bag according to claim 1, characterized in that the weight ratio of each material is: Semen Cassiae 100˜300 parts, chrysanthemum 50˜150 parts, lotus leaf 200˜400 parts and folium mori 50˜180 parts.

3. The Semen Cassiae-containing tea bag according to claim 2, characterized in that the tea bag also comprises following raw materials in parts by weight: rose 50˜150 parts, licorice 50∥150 parts, and herba houttuyniae 50˜180 parts.

4. The Semen Cassiae-containing tea bag according to claim 3, characterized in that the rose is Rosa rugosa cv.Plena.

5. The Semen Cassiae-containing tea bag according to claim 4, characterized in that the tea bag comprises following raw materials in parts by weight: Semen Cassiae 150 parts, chrysanthemum 50 parts, lotus leaf 220 parts, folium mori 100 parts, Rosa rugosa cv.Plena 75 parts, licorice 80 parts and herbs houttuyniae 180 parts.

6. A preparation method for Semen Cassiae-containing tea bag according to any one of claim 1, wherein the method comprises the following steps:
(1) sorting: taking fresh raw materials and cleaning, drying with 30˜50 DEG C hot wind, and removing visible impurities, the impurities content does not exceed 5% weight ratio;
(2) grinding: grinding the raw materials after step (1) by 40 meshes and obtaining a powdery mixture;
(3) sieving: sieving the powdery mixture, so that 95% weight ratio of the mixture can reach 40 meshes, and no more than 5%˜10% weight ratio of the mixture reach 60 meshes;
(4) sterilizing: placing a sieved powdery mixture into a material tray, and performing ozone sterilizing for 30 minutes, herein the material thickness in the material tray does not exceed 5 cm;
(5) weighing: weighing according to weight raio of each material in tea bag;
(6) mixing: mixing for 30 minutes in three dimensional mixer
(7) packing: using the integrative machine of tea bag to pack by direct forming method, each packing unit is 2.5 g.

7. The preparation method for Semen Cassiae-containing tea bag according to claim 6, characterized in that the packaged tea bag comprises following raw materials in parts by weight Semen Cassiae 100˜300 parts, chrysanthemum 50˜150 parts, lotus leaf 200˜400 parts, folium mori 50˜180 parts, Rosa rugosa cv.Plena 50˜150 parts, licorice 50˜150 parts and herba houttuyniae 50˜180 parts.

8. The preparation method for Semen Cassiae-containing tea bag according to claim 6, characterized in that a vacuum roasting step is added between the step (3) sieving and step (4) sterilizing, in the vacuum roasting, vacuum degree is 0.1˜1 MPa, and temperature is 50˜60 DEG C.

9. The preparation method for Semen Cassiae-containing tea bag according to claim 8, characterized in that a freezing step is added between the step (1) sorting and the step (2) grinding, the freezing step is to freeze the sorted fresh raw materials in step (1) for 3 h under minus 10˜minus 6 DEG C.

10. The preparation method for Semen Cassiae-containing tea bag according to claim 8, characterized in that after vacuum roasting, moisture content of materials is lower than 6%.
